(19) United States
(12) Patent Application Publication
(10) Pub. No.: US 2014/0114114 A1
(43) Pub. Date: Apr. 24, 2014

(54) Systems and methods for the management and provision of radiotherapy

(71) Applicant: Elekta AB (publ), Stockholm (SE)

(72) Inventor: Kevin Brown, Horsham (GB)

(73) Assignee: Elekta AB (publ), Stockholm (SE)

(21) Appl. No.: 14/138,722

(22) Filed: Dec. 23, 2013

Related U.S. Application Data
(63) Continuation of application No. PCT/EP2011/003170, filed on Jun. 28, 2011.

(51) Int. Cl. A61N 5/10

(52) U.S. Cl. CPC A61N 5/1031; A61N 5/1036; A61N 5/1081; A61N 5/1071
USPC 600/1

(57) ABSTRACT

Methods and apparatus for the management and provision of radiotherapy are described in which values for dosimetric parameters are re-evaluated just prior to treatment, on the basis of the treatment plan as loaded into the radiotherapy apparatus, and during treatment, on the basis of monitored machine parameters. By displaying dosimetric parameters rather than complicated machine parameters, the technician operating the radiotherapy apparatus is able to monitor the dose provided to a patient undergoing therapy. Misadministration of radiation as a result of corrupted data or corrupted control signals are prevented.

This application is a continuation of co-pending Patent Cooperation Treaty Application PCT/EP2011/003170, filed Jun. 28, 2011, which incorporated herein by reference in its entirety.

### TECHNICAL FIELD

The present invention relates to systems and methods for the management and provision of radiotherapy.

### BACKGROUND ART

Prior to beginning a course of radiotherapy, volumetric images of the patient, and specifically the target region, need to be obtained so that a plan for the treatment can be constructed. The aim of the treatment plan is to establish how to apply the radiotherapy to the patient so that the target region receives the desired, lethal dose, whilst the surrounding healthy tissue receives as little dose as possible.

Radiotherapy is often delivered by a system based on a linear accelerator (linac), which produces a beam of high-energy x-rays and directs this toward a patient. The patient typically lies on a couch or patient support, and the beam is directed toward the patient from an offset location. During treatment, the beam source is rotated around the patient while keeping the beam directed toward the target point. The result is that the target remains in the beam at all times, but areas immediately around the target are only irradiated briefly by the beam during part of its rotation. By positioning (for example) a tumor at the isocenter of the beam, the dose to the tumor is maximized whilst the dose to surrounding healthy tissue is reduced. In addition, the cross-section of the beam can be varied by way of a range of types of collimator, such as the so-called “multi-leaf collimator” (MLC) illustrated in European Patent 0214314, which is incorporated herein by reference in its entirety. These can be adjusted during treatment so as to create a beam whose cross-section varies dynamically as it rotates around the patient. Other aspects of the radiotherapy apparatus can also be varied during treatment, such as the speed of rotation of the source and the dose rate. Thus, there are a large number of variables offered by the apparatus in order to tailor the radiation dose that is delivered to the patient.

The volumetric images are therefore analyzed to identify a target region into which a minimum dose is to be delivered, any sensitive regions such as functional organs for which a maximum dose must be observed, and other non-target regions into which the dose is to be generally minimized. This three-dimensional map must then be used to develop a treatment plan, i.e. a sequence of source movements, collimator movements, and dose rates which result in a three-dimensional dose distribution that (a) meets the requirements as to maximum and minimum doses etc., and (b) is physically possible, e.g. does not require the source to rotate around the patient faster than it is physically capable.

This can be expressed as a mathematical problem in which the overall dose to healthy tissue must be minimized, subject to constraints as to the maximum dose to sensitive regions, the minimum dose to the target, and the various machine constraints such as the maximum rotation speeds, possible MLC shapes, etc. Although complex, the mathematical problem can be solved by one of a range of techniques (with varying efficiency) but this does require significant computing time.

The resulting plan is predicted to deliver a certain dose distribution which is reviewed and approved by the clinician, usually by comparing its dosimetric parameters (i.e. clinical metrics) to the values that are defined by the physician or protocol as acceptable. These dosimetric parameters are usually attributes of the ‘dose volume histograms’ (DVHs) derived from the plan. They consist of uniformity, maximum dose, minimum dose etc. The treatment plan is represented by a large number of machine parameters (for example, relevant to control of the linac) which once approved are made available to the linac for treatment.

### SUMMARY

Just prior to treatment, the treatment plan is loaded into the radiotherapy apparatus, and the radiotherapist is presented with the large number of machine parameters. Historically these were intended to help the radiotherapist have confidence that the correct treatment was about to be delivered to the patient and, once it had begun, that the treatment was being delivered correctly.

This approach was successful when the number of machine parameters was small (i.e. about ten). However, the techniques used in the delivery of radiotherapy have become increasingly complicated and the number of machine parameters is now often over a thousand. Presented with such a wealth of information, it is no longer possible for the radiotherapist to be able to determine whether or not the information displayed corresponds to the correct treatment. In fact, there have been a number of reported incidents where the radiotherapy control apparatus had the incorrect information but the user was unable to determine that fact from the parameters that were presented to him. Consequently, the patients were mistreated.

The present invention therefore provides, in a first aspect, a method of radiotherapy management, comprising: generating a treatment plan for the delivery of radiotherapy treatment to a patient, said treatment plan comprising values for a plurality of machine parameters associated with a radiotherapy apparatus; storing said treatment plan in a memory device; loading data corresponding to said treatment plan from said memory device into a device for controlling said radiotherapy apparatus; prior to treatment, calculating from said data expected values for one or more dosimetric parameters as a result of executing said treatment plan; and displaying said expected values to a user of said radiotherapy apparatus. The user of the radiotherapy apparatus (e.g. a technician) can thereafter take appropriate action if the expected values differ significantly from the norm. Alternatively, this can be part of an automated process. For example, in one embodiment, the method further comprises: comparing said expected values to acceptable values for said one or more dosimetric parameters; and on the basis of said comparison, allowing treatment to proceed or preventing said treatment from proceeding. The acceptable values and the expected values can be presented together to enable an easy comparison. The acceptable values in this case may be those values defined as safe maxima for the dosimetric parameters in question.

In an embodiment, the method further comprises: prior to storing said treatment plan, calculating from said treatment plan planned values for one or more dosimetric parameters as a result of executing said treatment plan; and displaying said planned values to a user of said radiotherapy apparatus. Thus, in this embodiment the planned values are shown to a user of the radiotherapy apparatus at the time of generating the treatment plan; the expected values (calculated on the basis of the stored treatment plan) are calculated just prior to treatment. The present invention also provides a control device for a radiotherapy system, comprising a processor and an input/output interface for communicating with said radiotherapy system, the processor being adapted to: access data corresponding to a radiotherapy treatment plan from a memory device; calculate from said data expected values for one or more dosimetric parameters as a result of executing said treatment plan; display said expected values to a user of said radiotherapy apparatus; and generate and transmit control signals to said radiotherapy apparatus, corresponding to said treatment plan, via said input/output interface.

In a second aspect, the present invention provides a method of radiotherapy management, comprising: generating a treatment plan for the delivery of radiotherapy treatment to a patient, said treatment plan comprising values for a plurality of machine parameters associated with a radiotherapy apparatus; storing said treatment plan in a memory device; loading data corresponding to said treatment plan from said memory device into a device for controlling said radiotherapy apparatus; during treatment, detecting values for said plurality of machine parameters and calculating therefrom values for one or more dosimetric parameters; and displaying said calculated values to a user of said radiotherapy apparatus.

In this aspect, therefore, the dosimetric parameters are calculated during treatment and thus correspond to the dose actually being delivered to the patient. Again, the user of the apparatus can either make a decision to halt the treatment on the basis of the information shown to him, or the process can take place automatically. Thus, in one embodiment, the method further comprises: comparing said calculated values to acceptable values for said one or more dosimetric parameters; and on the basis of said comparison, allowing treatment to continue or preventing said treatment from continuing. The acceptable values can be displayed with the calculated values, to enable an easy comparison to be made.

In an embodiment, the method further comprises: prior to storing said treatment plan, calculating from said treatment plan planned values for one or more dosimetric parameters as a result of executing said treatment plan; and displaying said planned values to a user of said radiotherapy apparatus. Thus, in this embodiment the planned values are shown to a user of the radiotherapy apparatus at the time of generating the treatment plan; the expected values (calculated on the basis of the detected values for the machine parameters) are calculated during treatment.

In this aspect, there is also provided a control device for a radiotherapy system, comprising a processor and an input/output interface for communicating with said radiotherapy system, the processor being adapted to: access data corresponding to a radiotherapy treatment plan from a memory device; generate and transmit control signals to said radiotherapy apparatus, corresponding to said treatment plan, via said input/output interface; receive feedback signals from said radiotherapy apparatus, corresponding to values for a plurality of machine parameters, via said input/output interface; calculate from said plurality of machine parameters expected values for one or more dosimetric parameters; and display said expected values to a user of said radiotherapy apparatus.

In all of the above embodiments, the plurality of machine parameters may comprise one or more parameters selected from: an angle of rotation of a gantry of the radiotherapy apparatus, positions of leaves in a multi-leaf collimating apparatus, a position of a collimating wedge, an energy of the beam of radiation. The one or more dosimetric parameters may be selected from: dose volume histograms, a maximum dose.

If there has been a fault where the linac parameters have been corrupted in a significant way since the TPS first calculated them, then this technique would display the incorrect dose and incorrect treatment could be prevented.

### BRIEF DESCRIPTION OF THE DRAWINGS

FIG. 1 shows a system according to embodiments of the present invention.

FIG. 2 shows a flowchart of a method according to embodiments of the present invention, as performed in a treatment planning phase.

FIG. 3 shows a flowchart of a method according to embodiments of the present invention, as performed just prior to and during treatment.

### DETAILED DESCRIPTION

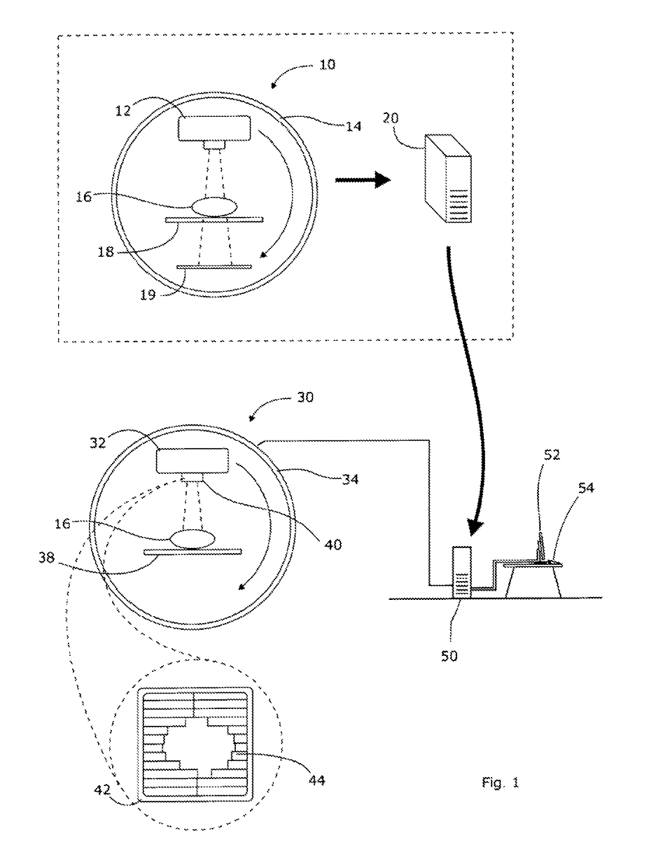

FIG. 1 shows a system according to embodiments of the present invention. Generally, the system comprises an imaging apparatus 10 and associated treatment planning apparatus 20, as well as a radiotherapy apparatus 30 and associated control device 50. Each of these components will be described in greater detail below. However, the person skilled in the art will appreciate that in practice there may be a degree of overlap in the functionality of the various apparatuses. For example, although they are illustrated separately in FIG. 1, a single apparatus may be able to perform both imaging and therapy functions. The present invention is therefore not limited solely to the embodiment as illustrated.

The imaging system 10 and the treatment planning apparatus 20 are used in conjunction to produce a treatment plan for providing radiotherapy to a patient. Any imaging apparatus may be used as part of this process (e.g. magnetic resonance imaging, computed tomography, ultrasound, etc.). The illustrated embodiment shows a CT apparatus 10, in which an x-ray source 12 generates a beam of imaging radiation (typically having an energy in the kilovoltage range). The source is mounted on a rotatable gantry 14, which is able to rotate around a patient 16 positioned on a support apparatus 18. A detector 19 is mounted on the gantry 14 diametrically opposite the source 12, and can thus detect the radiation after attenuation and scattering by the patient 16. A plurality of projection images are acquired at different angles of rotation, and these may be combined to reconstruct a three-dimensional image of the patient (or rather the area of interest in the patient). There are a large number of variables offered by the radiotherapy apparatus in order to tailor the radiation dose that is delivered to the patient (the exact number will depend on the nature of the radiotherapy apparatus). A treatment planning apparatus 20 (in practice a computing device implemented in hardware, software, or a combination of the two) is therefore provided, to generate a treatment plan and so control the radiotherapy apparatus 30 to provide a desired level of radiation dose to the patient.

Volumetric images of the patient are analyzed to identify a target region into which a minimum dose is to be delivered, any sensitive regions such as functional organs for which a maximum dose must be observed, and other non-target regions into which the dose is to be generally minimized. This three-dimensional map is then used to develop a treatment plan, i.e. a sequence of source movements, collimator movements, and dose rates which result in a three-dimensional dose distribution that (a) meets the requirements as to maximum and minimum doses (etc.) and (b) is physically possible, e.g. does not require the source to rotate around the patient faster than it is physically capable. This can be expressed as a mathematical problem in which the overall dose to healthy tissue must be minimized, subject to constraints as to the maximum dose to sensitive regions, the minimum dose to the target, and the various machine constraints such as the maximum rotation speeds, possible MLC shapes, etc. Although complex, the mathematical problem can be solved by one of a range of techniques (with varying efficiency). Various suitable techniques will be known to those skilled in the art, such as 2-phase ε-constraint (2psc) or ec, and the invention is not limited to any particular one.

Radiotherapy apparatus 30 is provided to implement the treatment plan. The radiotherapy system illustrated in FIG. 1 is generally indicative of the type of radiotherapy apparatus which may be employed in methods according to the present invention; however, radiotherapy may be provided by a range of different equipment, having different variables and constraints. The present invention is applicable to all types of radiotherapy, and radiotherapy systems.

A radiation head comprises a source of ionizing radiation 32 having sufficient energy to produce a therapeutic effect in the patient (i.e. generally in the megavoltage range), and a collimating device 40 to collimate that radiation into a beam of desired shape. The patient 16 is again positioned on a suitable support apparatus 38. The radiation head 32 is mounted on a rotatable gantry 34 such that, in operation, the radiation beam is directed towards a target region in the patient as from a number of different angles. By positioning the target region at or near the rotation axis of the gantry 34, the radiation beam intersects the target region at each angle of rotation, but passes through the surrounding tissue only momentarily. In this way, collateral damage to healthy tissue as a result of the treatment can be reduced.

The dashed-line projection in FIG. 1 shows a beam's eye view of the collimating device 40 in operation. A housing 42 defines a radiation field through which the radiation beam passes. In the illustrated embodiment, two banks of opposing leaves 44 are coupled to the housing 42 and extend across the radiation field to a greater or lesser extent as required. Each leaf is relatively thin in one direction, but relatively long in its direction of travel across the radiation field, and relatively deep in a direction parallel to the radiation beam axis (i.e. into the page in FIG. 1). The depth of the leaf, together with the choice of a manufacturing material having high atomic number (such as tungsten), acts to effectively block that part of the radiation field, preventing radiation from passing through. Each leaf is individually controllable to take any position in the range from falling outside the radiation field to extending fully across the radiation field, and thus the plurality of leaves can be controlled to define collectively a radiation beam having a desired cross-sectional shape (for example, to match the shape of a tumor or other target within the patient). This type of device is known as a multi-leaf collimator (MLC). Other collimating devices are known, however (such as binary collimators and block collimators), and the present invention is equally applicable to radiotherapy systems employing these types of devices.

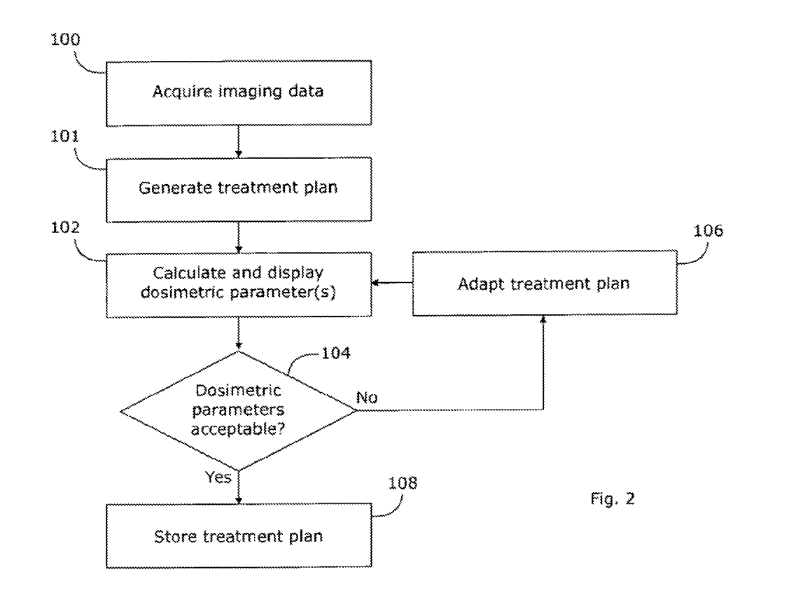

It will be apparent to the reader and to those skilled in the art that each course of radiotherapy involves control of a huge number of variables. Depending on the type of radiotherapy system employed, the variables may include: rotation angle of the radiation head; positions of the MLC leaves (or other collimating elements); energy of the beam; position of the “wedge” (an absorptive collimating element used to reduce skin dose in some methods of treatment); the type of treatment being delivered (e.g. electron therapy or x-ray therapy, etc); and the patient position. The treatment plan provides values for these machine parameters, controlling each element of the radiotherapy apparatus to deliver therapy which is appropriate to the patient's needs. A control apparatus 50 is therefore provided, into which the treatment plan is loaded, and from which the radiotherapy apparatus 30 is controlled. The control apparatus 50 may be associated with a display means (e.g. a monitor) 52, and a means for receiving input (e.g. a keyboard) 54. The method of operation of the imaging system 10 and the treatment planning apparatus 12 will now be described with reference to FIG. 2, which shows a flowchart according to embodiments of the present invention.

The method begins in step 100, in which the imaging apparatus 10 is employed to generate imaging data of a region of interest in the patient. The imaging data is provided to the treatment planning apparatus, and a treatment plan is generated in step 101. The treatment plan comprises values for a number of machine parameters, including one or more of: rotation angle of the radiation head; positions of the MLC leaves (or other collimating elements); energy of the beam; position of the “wedge” (an absorptive collimating element used to reduce skin dose in some methods of treatment); the type of treatment being delivered (e.g. for treatment of breast cancer, prostate cancer, etc.); the type of ionizing radiation used (e.g. electron therapy or x-ray therapy, etc.); and the patient position. Various suitable techniques will be known to those skilled in the art, such as 2-phase ε-constraint (2pεc) or εC, and the invention is not limited to any particular one.

In step 102, values for one or more dosimetric parameters resulting from the treatment plan are calculated and displayed to a qualified clinician. For example, the dose distribution as a result of the treatment plan can be calculated and displayed to the clinician; however, values other dosimetric parameters may be calculated and displayed as well or instead, e.g. dose volume histograms, a maximum dose, etc.

In step 104, the clinician determines if these values are acceptable, i.e. if they deliver a safe and appropriate level of radiation dose to the patient. If not, the plan is adapted in step 106 and the values recalculated and re-displayed. If the values are acceptable, the plan is approved and stored (step 108) in a storage device, i.e. a memory.

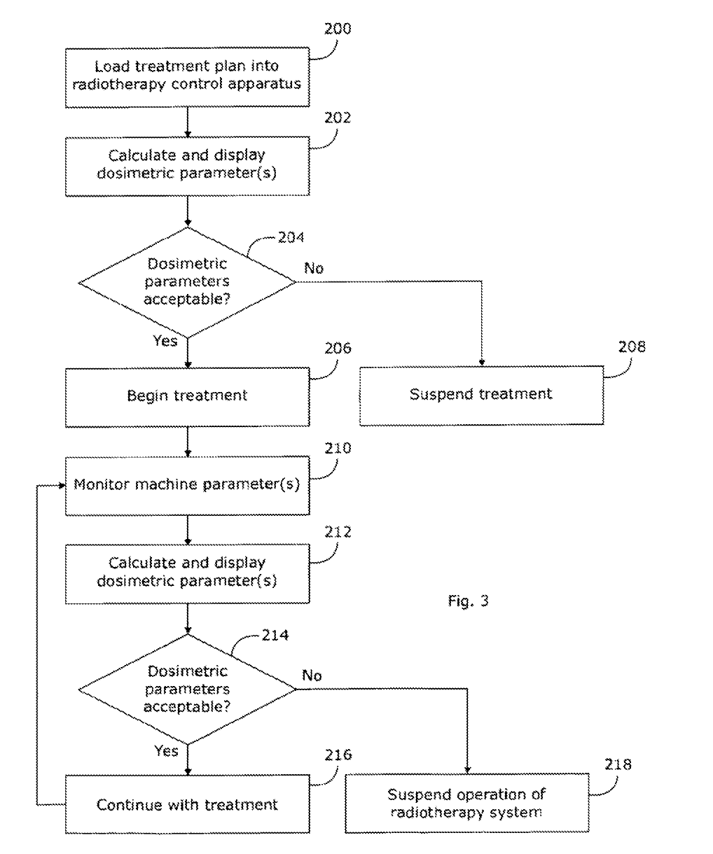

Treatment planning is a complex process, and plans can therefore take a considerable amount of time to generate even using modern processors. It is common for patients to wait one or several days before the treatment planning is complete and a window becomes available to use the radiotherapy apparatus. The method according to embodiments of the present invention has therefore been split into two phases: the treatment planning phase, described above; and the treatment phase, to be described below. FIG. 3 is a flowchart of a method in this latter phase, according to embodiments of the present invention.

The method begins in step 200, in which the treatment plan approved in step 108 is loaded into the control device 50 from memory. As with any memory, it is possible that the data may have become corrupted in saving the treatment plan to memory, storing the treatment plan in memory, or loading the treatment plan from memory. Given the large number of machine parameters specified in the treatment plan, it is difficult to notice an error in the treatment plan loaded into the control device.

In step 202, the radiotherapy control device 50 calculates values for one or more dosimetric parameters on the basis of the treatment plan loaded in step 200, and displays them to the user of the radiotherapy system (i.e. a technician). Note that the technician in the treatment phase will not in general be the same person as the clinician in the treatment planning phase. As before, values for several dosimetric parameters may be shown in this step. For example, dose volume histograms or a maximum dose may be calculated and shown to the technician. In an embodiment, acceptable values for the dosimetric parameters are shown in conjunction with the calculated values for these parameters. In one embodiment, the user interface is a simple graphical display of the dose to be delivered. This graphical display could include relevant clinical metrics and possibly graphics of the dose volume histogram, enabling the user or the computer to determine quickly and easily whether or not the machine parameters of the loaded treatment plan are about to deliver a dose distribution which is consistent with the physician's original intent. Dose volume histograms are based on the dose that is to be delivered and the definition of the patient's anatomy. In one embodiment, therefore, the patient's anatomy can be derived from the imaging information acquired during the treatment planning stage (step 100). In other embodiments, the patient may be imaged again at the time of treatment (i.e. during step 202 for example) and a new determination made of the patient's anatomy at that time. In step 204, therefore, it is determined whether the calculated values for the dosimetric parameters are acceptable (for example by comparing them to the acceptable values). This step may be performed by the technician, or automatically by the control device 50. If the calculated values are not acceptable, the treatment can be suspended in step 208.

If the calculated values are acceptable, the treatment is allowed to begin in step 206.

In further embodiments of the present invention, this process substantially continues during the radiotherapy treatment, based on measured values of the machine parameters rather than the treatment plan directly. Control signals sent from the control device 50 to the radiotherapy apparatus 30 are liable to corruption, and the controlled components of the radiotherapy apparatus itself are liable to mechanical failure. Thus, in step 210, the machine parameters specified as part of the treatment plan are monitored during the treatment. That is, the actual machine parameters are detected and fed back to the control device 50. The control device 50 calculates values for the one or more dosimetric parameters discussed above, and displays them to the technician operating the apparatus 30 (step 212). The technician or the control device then determines whether the actual values of the dosimetric parameters are acceptable in step 214 (i.e. whether they are sufficiently similar to defined acceptable values, or whether they are sufficiently similar to the values calculated in steps 104 and 202). If the values are not acceptable, the operation of the radiotherapy system 30 may be suspended in step 218. If acceptable, the treatment can be allowed to continue (step 216), and the machine parameters continue to be monitored.

The present invention thus provides methods and apparatus for the management and provision of radiotherapy, in which values for dosimetric parameters are re-evaluated just prior to treatment (on the basis of the treatment plan as loaded into the radiotherapy apparatus) and during treatment (on the basis of monitored machine parameters). By displaying dosimetric parameters rather than complicated machine parameters, the technician operating the radiotherapy apparatus is able to monitor the dose provided to a patient undergoing therapy. Misadministrations of radiation as a result of corrupted data or corrupted control signals are prevented.

It will of course be understood that many variations may be made to the above-described embodiment without departing from the scope of the present invention.

What is claimed is:

1. A method of radiotherapy management, comprising:
generating a treatment plan for the delivery of radiotherapy treatment to a patient, said treatment plan comprising values for a plurality of machine parameters associated with a radiotherapy apparatus;
storing said treatment plan in a memory device;
loading data corresponding to said treatment plan from said memory device into a device for controlling said radiotherapy apparatus;
prior to treatment, calculating from said data expected values for one or more dosimetric parameters as a result of executing said treatment plan; and
displaying said expected values to a user of said radiotherapy apparatus.

2. The method according to claim 1, further comprising:
comparing said expected values to acceptable values for said one or more dosimetric parameters; and
on the basis of said comparison, allowing treatment to proceed or preventing said treatment from proceeding.

3. The method according to claim 2, further comprising:
displaying said acceptable values with said expected values.

4. The method according to claim 1, further comprising:
prior to storing said treatment plan, calculating from said treatment plan planned values for one or more dosimetric parameters as a result of executing said treatment plan; and
displaying said planned values to a user of said radiotherapy apparatus.

5. A method of radiotherapy management, comprising:
generating a treatment plan for the delivery of radiotherapy treatment to a patient, said treatment plan comprising values for a plurality of machine parameters associated with a radiotherapy apparatus;
storing said treatment plan in a memory device;
loading data corresponding to said treatment plan from said memory device into a device for controlling said radiotherapy apparatus;
during treatment, detecting values for said plurality of machine parameters and calculating therefrom values for one or more dosimetric parameters; and
displaying said calculated values to a user of said radiotherapy apparatus.

6. The method according to claim 5, further comprising:
comparing said calculated values to acceptable values for said one or more dosimetric parameters; and
on the basis of said comparison, allowing treatment to continue or preventing said treatment from continuing.

7. The method according to claim 6, further comprising:
displaying said acceptable values with said calculated values.

8. The method according to claim 5, further comprising:
prior to storing said treatment plan, calculating from said treatment plan planned values for one or more dosimetric parameters as a result of executing said treatment plan; and
displaying said planned values to a user of said radiotherapy apparatus.

9. The method according to claim 5, wherein said plurality of machine parameters comprises one or more parameters selected from: an angle of rotation of a gantry of the radiotherapy apparatus, positions of leaves in a multi-leaf collimating apparatus, a position of a collimating wedge, an energy of the beam of radiation.

10. The method according to claim 5, wherein said one or more dosimetric parameters are selected from: dose volume histograms, a maximum dose.

11. A control device for a radiotherapy system, comprising a processor and an input/output interface for communicating with said radiotherapy system, the processor being adapted to:
access data corresponding to a radiotherapy treatment plan from a memory device;
calculate from said data expected values for one or more dosimetric parameters as a result of executing said treatment plan;
display said expected values to a user of said radiotherapy apparatus; and
generate and transmit control signals to said radiotherapy apparatus, corresponding to said treatment plan, via said input/output interface.

12. A control device for a radiotherapy system, comprising a processor and an input/output interface for communicating with said radiotherapy system, the processor being adapted to:
access data corresponding to a radiotherapy treatment plan from a memory device;
generate and transmit control signals to said radiotherapy apparatus, corresponding to said treatment plan, via said input/output interface;
receive feedback signals from said radiotherapy apparatus, corresponding to values for a plurality of machine parameters, via said input/output interface;
calculate from said plurality of machine parameters expected values for one or more dosimetric parameters; and
display said expected values to a user of said radiotherapy apparatus.
